(19) United States
(12) Patent Application Publication
(10) Pub. No.: US 2009/0204127 A1
(43) Pub. Date: Aug. 13, 2009

(54) SYSTEM AND METHOD FOR TYING SURGICAL KNOTS

(75) Inventor: M. Mary Sinnott, Logan, UT (US)

Correspondence Address: MEDICINELODGE INC., 124 South 600 West, LOGAN, UT, 84321, US

(73) Assignee: MEDICINELODGE, INC., Logan, UT (US)

(21) Appl. No.: 12/364,020

(22) Filed: Feb. 2, 2009

Related U.S. Application Data
(60) Provisional application No. 61/025,026, filed on Jan. 31, 2008.

(51) Int. Cl. A61B 17/04

(52) U.S. Cl. 606/139; 606/148

(57) ABSTRACT

A system and method for passing a suture through a tissue and tying a surgical knot. The system may comprise a suture and a first and a second arm. The first arm may comprise a needle and the second arm may comprise a tube configured to allow the needle of the first arm to pass through. One end of the suture may be wrapped around the tube and the other end of suture secured to the needle. The arms may engage. The needle and suture may pass through the tube and the portion of the suture wrapped around the tube. A knot is formed with the suture. The suture may be secured to the opposite site of the arm with the needle. The arms may disengage which may remove the needle from the tube. The knot may be tensioned by pulling on opposite ends of the suture in opposite directions.

### CROSS-REFERENCE TO RELATED APPLICATIONS

This application claims the benefit of the following, which is incorporated herein by reference:

Pending prior U.S. Provisional Patent Application No. 61/025,026 filed 31 Jan. 2008, which carries Applicants' docket no. MLI-74 PROV, and is entitled Knot Tier.

### BACKGROUND OF THE INVENTION

1. The Field of the Invention

The present invention relates generally to the tying of a surgical knot through use of a suture passer, and more particularly, to wrapping a suture around a tube of a suture passer to aid in tying surgical knots and to systems and methods of for tying surgical knots to secure tissues together.

2. The Relevant Technology

Suture passers are surgical instruments that are used in passing suture through tissue to repair damaged tissues. This is commonly done in repairing torn ligaments or tendons but can include a variety of tissues. Typically the surgeon uses a suture passer to thread a piece of suture through the tissue and then fasten the two ends of the suture by tying a knot.

One of the most common knots in surgical repair is a surgeon's knot. A surgeon's knot is essentially a double half hitch tied against another, opposite, single half hitch. One difficult aspect of tying a surgeon's knot is the looping of one working end of a suture around the other working end of the suture multiple times between the suture and the tissue. Other knots are just as difficult because of the small work area around the tissue.

Many surgical knots are performed simply by passing a suture through tissue using a needle to pierce the tissue and the surgeon manipulating the suture to form the desired knot without any device other than the surgeon's hands, the needle and the suture.

Some systems use implants with multiple channels. The suture is passed through at least two of these channels within the implant and then the suture is secured to the implant.

There is a need to have an instrument that can be used to tie a knot to a tendon or ligament, or through soft tissue. The instrument should tie at least a half hitch but could be configured to do more through passes if needed. The user should be able to load suture onto the instrument prior to passing the suture around or through the tissue.

As the above described techniques illustrate, the existing systems and procedures for suture passing combined with knot tying may not be as effective as desired.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 illustrates a side view of an instrument, with a handle portion, a shaft, a distal portion and a suture;

FIG. 2 illustrates a side view of the distal portion of the instrument of FIG. 1 with a pinched tissue, a first arm, a second arm and a suture with the arms in a disengaged position;

FIG. 3 illustrates a side view of the distal portion of the instrument of FIG. 1 with the first arm and the second arm in an engaged position with a needle and the suture passing through a boss with a bore running therethrough;

FIG. 4 illustrates a side view of the distal portion of the instrument of FIG. 1 with the first and the second arm in the disengaged position with the suture forming a knot;

FIG. 5 illustrates a side view of the suture of FIG. 1 tied in a first knot disengaged from the instrument of FIG. 1;

FIG. 6 illustrates a side view of the first knot of FIG. 5 with the suture tied in a second knot;

FIG. 7 illustrates a side view of an alternate embodiment of the distal portion of the instrument of FIG. 1 with a tissue positioned substantially parallel with a medial line of the distal portion.

### DETAILED DESCRIPTION OF THE PREFERRED EMBODIMENTS

The present invention relates to systems and methods for suture passing through tissue and knot tying for securing a suture to at least one tissue or securing at least two tissues together. Those of skill in the art will recognize that the following description is merely illustrative of the principles of the invention, which may be applied in various ways to provide many different alternative embodiments. This description is made for the purpose of illustrating the general principles of this invention and is not meant to limit the inventive concepts in the appended claims.

One embodiment of the present invention includes a suture passer instrument with two jaws and a suture. A first jaw comprises a boss defining a bore and a second jaw comprises a needle, with a slit. The slit provides passage of the suture to attach the suture to the needle. The bore provides a passage through the boss and the first jaw for the needle with the suture. A method of tying a knot while passing the suture through tissue includes wrapping a first end of the suture around the boss to form a loop and feeding a second end of the suture through the needle. The jaws of the suture passer instrument may be urged together and bite the tissue, or tissues, forcing the needle end of the suture to pass through the tissue(s) and through the boss. The needle protrudes from the other side of the opposite jaw and the second end of the suture is secured preventing withdrawal of the second end of the suture back through the bore. The first end and second end can then be looped around and tied together to complete a surgeon's knot. With the simultaneous passing of the suture through tissue and the formation of a double half hitch knot, fewer steps are required to secure the suture to the tissue as well as fewer steps to secure a first tissue to a second tissue.

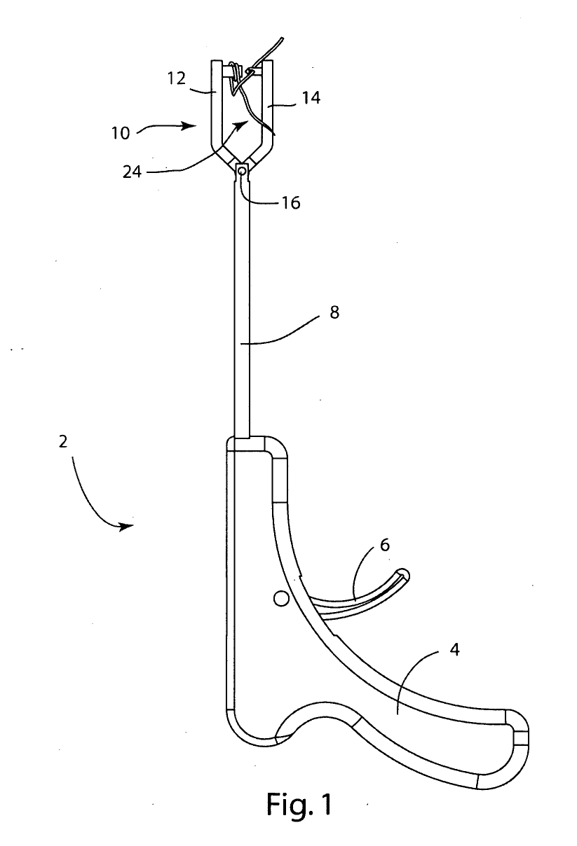

Referring to FIG. 1, a suture passer instrument 2 comprises a proximal handle portion 4, an intermediate portion 8 which may be a shaft, and a distal suture passer portion 10. The handle portion 4 is grippable by a user. The handle portion 4 includes an actuator 6 which may be a trigger or any other means known in the art for manipulating a surgical instrument. In FIG. 1, the trigger 6 protrudes from the handle portion 4 in a substantially downward direction; however, it will be appreciated that the trigger 6 may be positioned anywhere on the handle so as to provide the user with access to the trigger 6 for actuation of the suture passer portion 10. The shaft 8 extends longitudinally from the distal end of the handle portion 4. The suture passer 10 extends from the distal end of the shaft 8. The suture passer 10 may extend in any of multiple planes from the shaft 8.

The suture passer 10 comprises a first arm 12 and a second arm 14 connected at their proximal ends to a pivot member 16 such that at least one of the arms 12, 14 can move relative to the other when the trigger 6 is manipulated. The first arm 12 may move toward or away from the second arm 14 or vice versa, or both arms 12, 14 may move, when the trigger 6 is manipulated. In another embodiment of the invention, a first arm may be integral with the shaft and a second arm may be movable relative to the shaft and the first arm.

It will be appreciated that the instrument 2 may be comprised of many different pieces and these pieces may be comprised of many different materials which may include metals, metal alloys, semi-rigid plastics or polyurethane, among others.

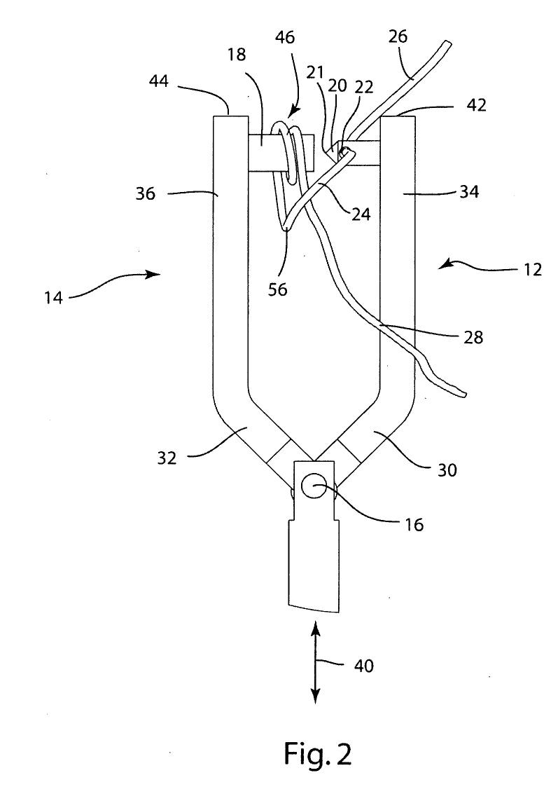

FIGS. 2-4 illustrate an embodiment of the suture passer with a suture, two arms connected at their proximal ends to the pivot member, a boss extending from one of the arms and a needle extending from the opposite arm.

Referring to FIG. 2, the first arm 12 and the second arm 14 extend from the pivot member 16. The two arms 12, 14 may extend substantially equidistant from the pivot member 16 and may be mirror images. In the embodiment of FIG. 2, a first proximal arm portion 30 of the first arm 12 may extend in a first direction away from a medial line 40 of the suture passer 10. A first distal arm portion 34 of the first arm 12 connected to the first proximal portion 30 may extend in a second direction substantially parallel to the medial line 40. A second distal arm portion 32 of the second arm 14 may extend in a third direction away from a medial line 40. A second distal arm portion 36 connected to the second proximal portion 32 may extend in the same second direction as the first distal arm portion 34 substantially parallel to the medial line 40 and substantially parallel to the first distal arm portion 34.

Alternate embodiments of the arms may include the first arm 12 extending in the first direction from the medial line 40 until reaching a first distal end 42 of the first arm 12 and the second arm 14 extending in the third direction from the medial line 40 until reaching a second distal end 44 of the second arm 14. In this configuration, the two arms 12, 14 are substantially mirror images of each other, forming a shape similar to a “Y.”

The first arm 12 may comprise a piercing member 20 which may be a needle, protruding nonparallel from the first distal portion 34 of the first arm 12. The needle may protrude toward the second arm 14 and/or toward the medial line 40. The needle 20 comprises a retention member 22 which may be an eye, hole or slit configured to retain a suture 24. The needle 20 may be cylindrical, conical, flat, or any other shape conducive to piercing tissue, retaining a suture and passing through a bore. The needle 20 may further comprise a point 21 configured to pierce tissue.

The second arm may comprise a boss 18 extending nonparallel to the second distal portion 36 of the second arm 14. The boss may protrude toward the first arm 12 or toward the medial line 40. The boss 18 comprises a bore 48 which may be a tube extending longitudinally through the boss 18 and also extending through the second distal portion 36 of the second arm 14. The bore 48 may be cylindrical but may be any shape that is conducive to receiving the needle 20 with the retained suture 24 and allowing passage therethrough.

The suture 24 comprises a first end or working portion 28 and a second end or working portion 26, wherein the first working portion 28 and the second working portion 26 may be contiguous. The first working portion 28 is wrapped around the boss 18, nonparallel to the second arm 14. The first working portion 28 encircles the boss 18, forming a first loop 46. The first working portion 28 is capable of encircling the boss 18 one time or multiple times and may form a helix of multiple loops. The second working portion 26 extends from the first loop 46 and passes through the retention member 22 and is retained therein. The second working portion 26 passes on top of or across the first working portion 28, and both working portions pass on top of or over a piece of tissue 50. A bight 56 is formed with the second working portion 26 between the first loop 46 encircling the boss and the retention member 22. The bight 56 may vary in size and length and may pass around a post (not pictured) at or near the handle portion 4 of the instrument 2.

In an alternate embodiment, the suture 24 may pass through a retaining feature of a suture anchor (not pictured) such that the first and second working portions 26, 28 extend from the suture anchor, and are positioned on the suture passer 10 as depicted herein. In this instance the bight 56 would pass through the suture anchor.

It will be appreciated that there are other possible ways to wrap a suture around a boss including, but not limited to, passing either one of the working portions 26, 28 through the loop 46 or passing both working portions 26, 28 through the loop 46 provided, however, that one of the working portions 26, 28 passes through the retention member 22.

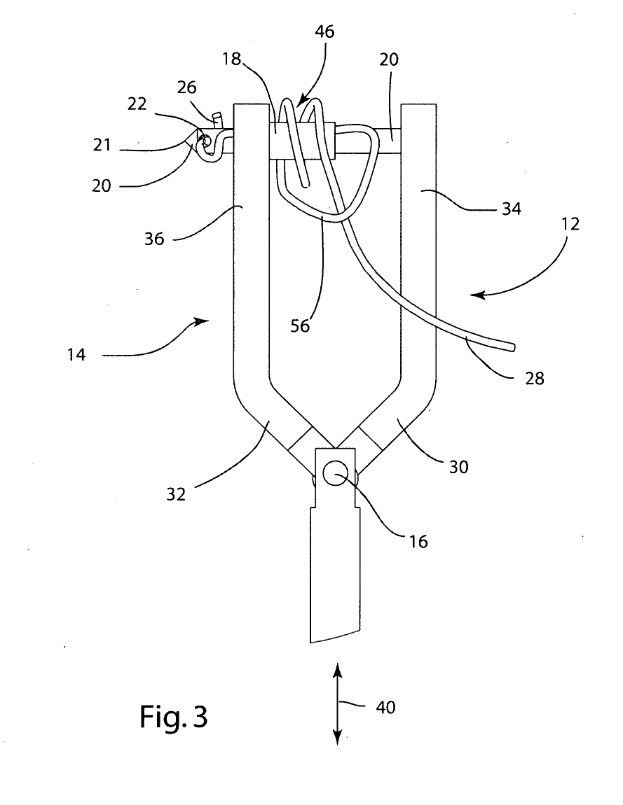

Referring to FIG. 3, the tissue 50 depicts a tissue folded or pinched to allow piercing of the tissue 50 and suture 24 to enter the tissue at one point, pass through or under the tissue and exit at a second point, forming two holes in the tissue. The tissue 50 is pierced when the actuator or trigger 6 is manipulated causing the arms 12, 14 to come together, whether it be the first arm 12 moving toward the second arm 14 or the second arm 14 moving toward the first arm 12 or both arms 12, 14 moving toward each other. The needle 20 pierces the tissue while retaining and passing the second working portion 26 of the suture through the piercing. The needle 20 passes through the bore 48 of the boss 18 to the lateral side of the second arm 14. The suture 24 forms a first knot 52. The second working end 26 may be withdrawn from the retention member 22 of the needle. The second working portion 26 of the suture 24 may be secured, at least temporarily, on the lateral side of the second arm 14 by any means known in the art so as to prevent withdrawal of the second working portion 26 of the suture 24 back through the loop 46.

An alternate embodiment (not pictured) may include securing the needle 20 along with the second working portion 26 of the suture on the lateral side of the second arm 14 and disengaging the needle 20 from the first arm 12.

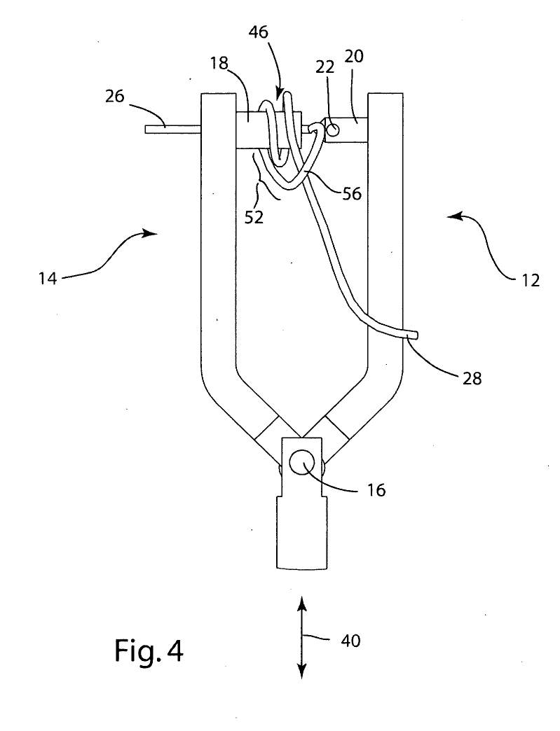

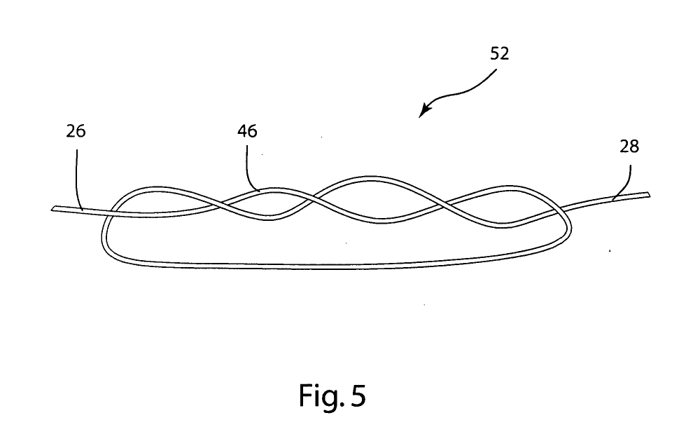

Referring to FIGS. 4 and 5, the trigger 6 is released to allow the arm or arms 12, 14 to return to their previous, disengaged positions apart from one another leaving the first knot 52. The suture passer 10 may be disengaged from the suture 24 completely. The first knot 52 that was formed from the passage of the second working portion 26 through the tissue 50 and the loop 46 may be tightened by tensioning of the first working portion 28 and the second working portion 26 by pulling or advancing each working portion 26, 28 in opposite directions.

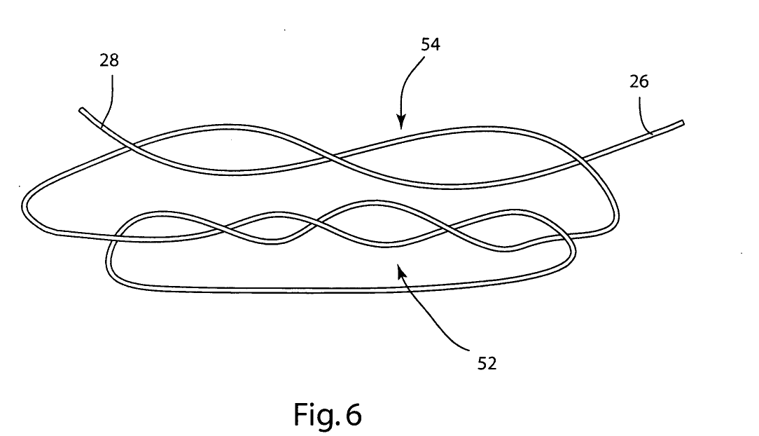

Referring to FIG. 6, to complete a surgeon's knot a user may tie a second knot 54 abutting the first knot 52. Completing a surgeon's knot may be done by tying a single half hitch or double half hitch knot 54 and tightening said knot 54 until secure, abutting the first knot 52 through tensioning of the first working portion 28 and second working portion 26. The second knot 52 may be tied manually, by using the suture passer 10, or by another means known in the art. A surgeon's knot is not the only knot capable of being tied with the disclosed invention but rather is illustrative to show a single embodiment. Other knots used within surgical procedures are encouraged and expected to be used with the disclosed system and/or method.

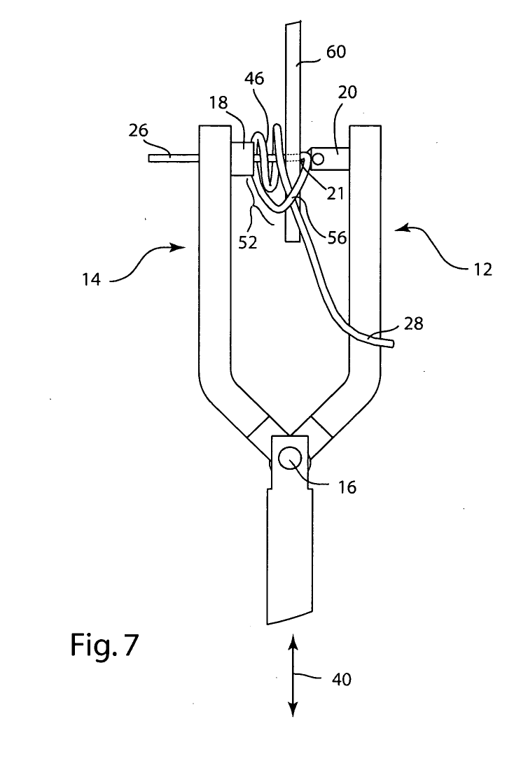

Referring to FIG. 7, an alternate embodiment is shown with a tissue 60 positioned longitudinally with the medial line 40 between the first arm 12 and the second arm 14 such that no fold is necessary, similar to how a grasper may be used to grab onto rotator cuff tissue. The tissue 60 is pierced in the same manner as previously described except that the piercing only creates a single hole rather than a double hole as when the tissue is “pinched.” The same methods for the previously disclosed embodiment apply to this embodiment as well.

It will be appreciated that multiple tissues may also be pierced, the suture passed through and a knot formed with a single action. These techniques may include two ends of one tissue or multiple ends of multiple tissues.

This system and/or method may also be used to tie knots around tissues including tendons and ligaments. The use of the system or method in securing a suture to a tendon or ligament may or may not pierce the tissue.

While this system and method is preferably performed with a suture, other likely flexible lines may be used which include nylon cord, polymer cord or wire.

Furthermore, the suture passer 10 may be embodied solely as the suture passer without a shaft 8, handle portion 4 or trigger 6 or any other added instrumentation.

The present invention may be embodied in other specific forms without departing from its spirit or essential characteristics. For example, above are described various alternative examples of suture passing, suture security and instrument mobility. It is appreciated that various features of the above-described examples can be mixed and matched to form a variety of other combinations and alternatives. It is also appreciated that this system should not be limited to simply to suture passage and knot tying. This system may also be used to secure a suture around a ligament, a tendon or other soft tissue. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for tying a surgical knot to tissue, the system comprising:
an instrument comprising:
a first arm;
a second arm connected to the first arm;
a piercing member protruding from the first arm, the piercing member comprising a retention feature; and
a boss protruding from the second arm along a direction non-parallel to the second arm, the boss comprising a bore extending therethrough, wherein the bore is shaped to receive the piercing member; and
a suture comprising a first working end and a second working end, a portion of the first working end encircling the boss to form a loop, wherein the second working end extends from the loop and is captured by the retention feature,
wherein the first arm is movable relative to the second arm to urge the piercing member through the tissue and into the bore such that the retention features carries the second end through the tissue and the loop to form a first knot.

2. The system of claim 1, wherein the portion of the first working end encircles the boss twice to form a helix comprising two loops.

3. The system of claim 1, wherein the piercing member comprises a needle.

4. The system of claim 1, wherein the retention feature comprises an eye.

5. The system of claim 1, wherein the first arm and the second arm are pivotably coupled together.

6. The system of claim 1, wherein the instrument further comprises:
a proximal portion grippable by a user; and
a distal portion, wherein the first and second arms, the piercing member, and the boss are incorporated into the distal portion.

7. The system of claim 6, wherein the proximal portion comprises a handle.

8. The system of claim 6, wherein the instrument further comprises a shaft formed between the proximal portion and the distal portion.

9. The system of claim 6, wherein the proximal portion comprises an actuator connected to at least one of the first arm and the second arm, wherein the actuator is actuable from the proximal portion to move the first arm relative to the second arm.

10. A method for tying a surgical knot to tissue, the method comprising:
encircling a portion of a first working end of a suture around a boss to form a loop, the suture further comprising a second working end extending from the loop, the boss protruding along a non-parallel direction from a second arm of an instrument, the boss comprising a bore extending therethrough;
capturing the second working end by a retention feature of a piercing member, the piercing member protruding from a first arm of the instrument connected to the second arm; and
moving the first arm relative to the second arm to urge the piercing member through the tissue and into the bore;
wherein, in response to motion of the piercing member through the tissue and into the bore, the retention feature carries the second working end through the tissue and the loop to form a first knot.

11. The method of claim 10, wherein encircling the portion of the first working end of the suture around the boss to form the loop further comprises encircling the portion around the boss at least two times to form a helix comprising at least two loops.

12. The method of claim 10, further comprising securing the second working end after it passes through the tissue and the loop to prevent the second working end from being withdrawn through the tissue and the loop.

13. The method of claim 10, further comprising pulling at least one of the first working end and the second working end after the second working end is carried by the retention feature through the tissue and loop to form the knot, to tighten the knot.

14. The method of claim 10, further comprising:
withdrawing the second working end from the retention feature after the retention feature has passed through the tissue and the loop; and
withdrawing the piercing member from the bore after the second working end has been withdrawn from the retention feature.

15. The method of claim 10, wherein the first arm and the second arm are pivotably coupled together, wherein moving the first arm relative to the second arm comprises pivoting the first arm relative to the second arm.

16. The method of claim 10, wherein the instrument further comprises a proximal portion comprising an actuator, the actuator connected to at least one of the first arm and the second arm, wherein moving the first arm relative to the second arm to urge the piercing member through the tissue and into the bore comprises actuating the actuator to move the first arm relative to the second arm.

17. The method of claim 10, further comprising:
tying a second knot with the first working end and the second working end after tying the first knot, the first and second knots forming a surgeon's knot.

18. A method for tying a surgical knot to tissue through the use of an instrument, the method comprising:
coupling a first loop of the suture to a first arm of the instrument;
coupling a second working end of the suture to a retention feature of a tissue piercing member of the instrument;
positioning the first arm and the tissue piercing member such that the tissue lies between the first loop and the tissue piercing member;
actuating the instrument to move the tissue piercing member toward the loop such that the tissue piercing member pierces the tissue and the retention feature carries the second working end through the tissue and through the first loop.

19. The method of claim 18, further comprising:
coupling a second loop of the suture to the first arm of the instrument; and
actuating the instrument to move the tissue piercing member toward the first and second loops such that the tissue piercing member pierces the tissue and the retention feature carries the second working end through the tissue and through the first and second loops.

20. The method of claim 18, further comprising securing the second working end after it passes through the tissue and the loop to prevent the second working end from being withdrawn through the tissue and the loop.

21. The method of claim 18, further comprising pulling at least one of the first working end and the second working end after the second working end is carried by the retention feature through the tissue and loop, to tighten the knot.

22. The method of claim 18, further comprising:
withdrawing the second working end from the retention feature after the retention feature has passed through the tissue and the loop; and
withdrawing the tissue piercing member through the tissue after the second working end has been withdrawn from the retention feature.

23. The method of claim 18, further comprising:
using the instrument to tie a second knot with the first working end and the second working end after tying the first knot, the first and second knots forming a surgeon's knot.
